(19) United States
(12) Patent Application Publication
(10) Pub. No.: US 2003/0131393 A1
(43) Pub. Date: Jul. 17, 2003

(54) Utensil for eliminating bare hand handling of sensitive material

(76) Inventor: Earl J. Votolato, Newport Beach, CA (US)

Correspondence Address: Kit M. Stetina, STETINA BRUNDA GARRED & BRUCKER, Suite 250, 75 Enterprise, Aliso Viejo, CA, 92656, US

(21) Appl. No.: 10/047,059

(22) Filed: Jan. 14, 2002

(51) Int. Cl. A61F 9/00; A41D 1/00

(52) U.S. Cl. 2/16; 2/158

(57) ABSTRACT

A utensil for handling sensitive material is disclosed. The utensil includes a pair of pockets for receiving a user's hand and being formed of a flexible material, wherein each of the pockets has an open end, a closed end and two sides. One side of each pocket is flat and arranged in opposition to the other, thereby forming opposing sides. A hinge joins together the pair of pockets at the opposing sides and at the open ends thereof. The hinge biases the pockets in a closed position such that the pair of pockets are separable by the user for gripping the sensitive material with the opposing sides at the closed ends of the pockets.

### CROSS-REFERENCE TO RELATED APPLICATIONS

(Not Applicable)

### STATEMENT RE: FEDERALLY SPONSORED RESEARCH/DEVELOPMENT

(Not Applicable)

### BACKGROUND OF THE INVENTION

The present invention generally relates to handling utensils and in particular to a one-piece gripping tool for handling sensitive material.

One example of sensitive material to be handled is food, whether raw or cooked. The production and serving of food in the home and retail environment requires a high degree of bare hand contact with the food prior to serving. Unfortunately, a drawback of this bare hand contact is that holding food directly with the hands allows dirt, chemicals, germs and the like to come into direct contact with the food. Although washing the hands provides a degree of protection from the risks associated with the unsanitary practice, there nevertheless remains a significant risk of detriment to one's health when the food is ingested. This is because all of the germs cannot easily be washed or scrubbed from a person's hands, especially the finger tips and the underside of the fingernails. Bacteria clings tenaciously via electrostatic forces to a person's skin and thus cannot be easily rubbed off or washed off. In addition, not all types of soap are capable of destroying all the bacteria on a person's skin. Moreover, many people do not take the time and effort to effectively wash their hands thoroughly prior to the handling of food.

Since a person's hands frequently come into contact with a variety of objects and body surfaces, this is consequently a significant way in which people can cross contaminate sensitive material such as food with germs and other food borne microbes. Another detriment resulting from this unsanitary practice is that many people use a variety of industrial chemicals, such as inks, solvents and the like, household chemicals such as cleaning compounds, insecticides and the like, all of which are known to be harmful if ingested but nevertheless are useful for their commercial or home use and thus remain in common use. Some of the chemicals that people handle become embedded in the skin of the hands and for this or other reasons remain on the skin even after washing, albeit perhaps in just trace amounts. Consequently, these chemicals can be transferred to the food and promptly served and ingested. This occurs to a much greater degree when preparing certain foods that commonly contain copious amounts of oil such as deep fried chicken, french fries, tacos, and the like. Since the oil acts to emulsify these chemicals (as well as dirt and other types of contaminants) it is prudent to remove them from the hands and not transfer them to food.

In an effort to address this problem, many types of utensils have been designed to handle items of sensitive material, such as food. Some of these utensils include a pair of pivotally or hingedly connected arms, the opposing ends of which may be brought together into contact with each other and thus are used to grip a piece of sensitive material. The opposing ends of many designs of such utensils include teeth to provide a means for gripping the sensitive material. An example of such a utensil is disclosed in U.S. Pat. No. 4,728,139 to Oretti. The Oretti utensil is a pair of tongs that have substantially similar length arms joined at their inner ends by an integral junction portion. The junction portion bears against a fulcrum block at the free end portions of the respective arms in order to relieve or eliminate bending stress on the junction. However, a primary disadvantage of the Oretti design is its complexity, which makes it more expensive to manufacture. In addition, the teeth of the arms are straight and thus do not provide full contact with many types of sensitive material.

Other such utensils have been specifically designed to hold certain types of food. An example of such a utensil is disclosed in U.S. Pat. No. 4,802,704 to Burns. The Burns utensil consists of two oppositely disposed members pivotally joined at one end to simulate retractable jaws, which are spring biased so that they are normally in an open position. The unjoined ends of the members are provided with opposing teeth for grasping a spare rib. One of the members is also provided with a ramp for guiding the spare rib into the jaws. Although the Burns holder can grasp the spare rib at only one location, its general dimensioning and wall structure act to retain the spare rib therebetween. However, a primary disadvantage with such holders is that they are suitable only for certain types of foods. In addition, as with the Oretti utensil, the Burns holder is somewhat complex in construction and thus not inexpensive to manufacture. Moreover, the hinge construction provides an area in which bacteria or other contamination may collect. Such contamination at the hinge also hampers rotational movement and makes it unsuitable for reuse (although it is not inexpensive to purchase) because it is difficult to clean.

Other utensils are designed to be of one piece construction. U.S. Pat. No. 3,934,915 to Humpa discloses a one piece pair of tongs composed of plastic and provided with rows of teeth at the upper and lower end portions thereof. However, a primary disadvantage with the Humpa tongs are that the rows of teeth are flat and thus not shaped to conform to chicken drumstick bones or other types of food, which have curved portions. Thus, the flat rows of teeth are able to grip the curved portion of the food, e.g., the bone, at only one location thereby resulting in a somewhat less than secure and rigid grasping utensil.

Some tong types of utensils have curved portions providing more secure gripping. Two examples of such utensils are disclosed in U.S. Pat. Nos. 4,577,900 and 4,877,280 to Milano and Chasen, respectively. The Milano tongs have convex gripping end surfaces and are used for picking up paper. The Chasen tongs are used for eating and have outer lateral knurled portions which are longitudinally curved to conform to the shape of the food or other things to be gripped. However, the rows of teeth positioned inside the lateral portions are not curved. Moreover, only the front of the longitudinal portions are knurled, thereby limiting the full grasping action of the tongs to foods gripped from the lateral sides. The front of the outer longitudinal portions is also straight rather than curved, thereby further limiting its full utility of grasping food from the lateral sides. In addition, the Chasen design utilizes two tong members joined together by a pin, and also has spring-loaded outer end portions. The design is not only complex, but has areas for connections that collect contamination and germs and which are difficult to properly clean. In addition, these connections can become clogged with particles, such as food, thereby impeding rotational movements of the tongs and compromising its utility.

More recent examples of utensils that attempt to overcome the problems stated above include disposable mitts or gloves made from plastic film such as Latex. These mitts, when used to handle foods, pose an even larger problem in that they provide a false sense of cleanliness to a user. A user feels that they don't have to change the gloves because if their hands are clean this translates into the thought that it is permissible to handle food. Also, many people are allergic to Latex products and have reactions to them.

Another more recent example of a handling utensil is disclosed in U.S. Pat. No. 4,188,055, entitled DISPOSABLE SHOVEL TONGS by Green. This patent discloses a disposable shovel for sanitary removal of obnoxious matter such as animal excrement or litter. The shovel tongs are made of a stiff disposable material, such as a biodegradable material. The tongs include finger insertion external glove-like pockets. This prior art utensil is designed for the specific purpose of sanitary removal and disposal of animal excrement. It is not suitable as a utensil for handling such sensitive material as food.

There is therefore a need for a utensil that eliminates all bare hand contact with sensitive material, such as foods, micro-electronic components, medical instruments, drugs, certain chemicals and the like. The utensil should be simple in construction, have no connection points. Furthermore, the utensil should be inexpensive to manufacture and relatively trouble free. Moreover, the utensil should be safe to use and easy to clean. What is also needed is such a utensil that provides enhanced versatility by allowing sensitive material to be safely handled off hot or cold surfaces and gripped from the front or lateral sides thereof. Moreover, a device is needed that provides firm and secure gripping of the material, thereby enabling enhanced dextrous control so as to allow safe cutting, slicing or moving of the material.

### BRIEF SUMMARY OF THE INVENTION

An aspect of the present invention may be regarded as a utensil that eliminates bare hand contact with sensitive material. The utensil includes a pair of opposing pockets for receiving a user's hand. Each of the pockets has an open end and a closed end. The pair of pockets are joined together at their open ends by a hinge. The hinge biases the pockets in a closed position such that the pair of pockets are separable by the user for gripping the sensitive material at the closed ends of the pockets.

The pair of pockets and hinge may be made from a flexible material having memory. The pair of pockets and hinge may be made from a flexible plastic material having memory, a flexible metal material having memory or a flexible paper material having memory.

The utensil may be made of a color designating a task designation. Different colors are used to designate different tasks.

The opposing surfaces of the pair of pockets may be flat. The opposing surfaces may be bent inward near the closed ends of the pair of pockets to form pincers that enable the handling of small articles.

### BRIEF DESCRIPTION OF THE DRAWINGS

The general purpose of this invention, as well as a preferred mode of use, its objects and advantages will best be understood by reference to the following detailed description of an illustrative embodiment with reference to the accompanying drawings in which like reference numerals designate like parts throughout the figures thereof, wherein:

FIG. 1 is a perspective view of the utensil of the present invention;

FIG. 2 is another perspective view of the utensil of the present invention in operation; and

FIG. 3 is a perspective view of the utensil of the present invention as it may typically be used by a food handler.

### DETAILED DESCRIPTION OF THE INVENTION

The present invention provides a utensil for eliminating bare hand handling of sensitive material. While the present application is described with reference to the handling of foods, it will be appreciated that the present invention can also be used for the handling of other sensitive materials such as micro-electronic components, medical instruments, drugs, certain chemicals and the like. The utensil is easy to use and provides a variety of safety features. The utensil eliminates bare hand contact with sensitive material. Where the material being handled is food, use of the utensil reduces the chance of food borne illness. Lacerations and cuts are also eliminated when the utensil is used to hold food during slicing or cutting with knives. Burns are also minimizes as the user is protected from hot foods and cooking surfaces. The utensil also eliminates the need for heat resistant gloves, spatulas and food handling utensils. The need for plastic films mitts or gloves, such as latex gloves, is eliminated.

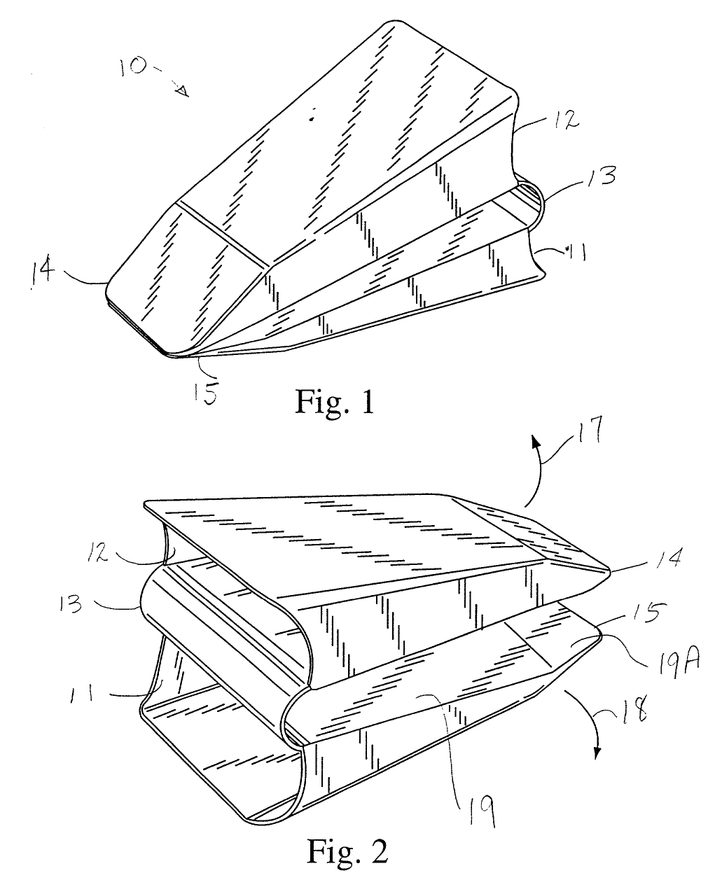

Referring now to the drawings and FIG. 1 in particular, a perspective view of a hands-off sensitive material handling utensil 10 of the present invention is shown. The utensil 10 is a one-piece construction of a molded flexible material, such as plastic, metal, paper, or some combination thereof. Preferably, the utensil is manufactured using a blow or injection molding process. However, it will be appreciated that other manufacturing methods may be used. A one-piece construction makes the utensil 10 inexpensive to manufacture. The selection of materials allow for the manufacture of a utensil 10 which can be easy to clean and sanitize. A pair of opposing pockets 11 and 12 are formed for receiving a user's thumb and fingers, respectively. A hinge 13 joins the pockets 11 and 12. The hinge biases the utensil 10 in a closed position as shown in FIG. 1. The hinge 13 is preferably integral and unitary with the pockets 11 and 12, and preferably made from a piece (or sheet) of plastic or suitable flexible material, which allows bending thereof so that the pockets 11 and 12 can move so as to open and close the utensil 10. The hinge 13 is preferably made of a material that has memory so that after the hinge is bent (for opening the utensil 10) it tends to revert back to its closed shape and position (as shown in FIG. 1). This allows the utensil 10 to be in a closed position when not in use. This also allows the utensil 10 to be easy to grasp by a user slipping their thumb into one pocket and their four remaining fingers into the other pocket. The springy characteristic of the hinge 13 is not of such a high degree that it requires exertion of a large amount of strength to open manually. The pockets 11 and 12 taper down to a pair of material grabbing tips 14 and 15, respectively.

FIG. 2 illustrates the utensil 10 in a partially open position. The two pockets 11 and 12 are moved by a user in the direction of arrows 17 and 18, against the bias of the hinge 13 attempting to close the tips 14 and 15 together. It is noted that the shape of the utensil 10 enables ease of storage, which does not require much space, because a plurality of such utensils may be nested together. The utensil 10 is also convenient to use and user friendly since it is easy to slip on and off and since it is biased in the normally closed position. Moreover, the simplicity of construction (e.g., from a single sheet of plastic) means that there are no small parts required that could fall off into the material being handled.

The inner or opposing surface 19 of each of the pockets 11 and 12 are substantially flat and a portion 19A of the opposing surfaces are bent inward so as to facilitate the handling of small articles. As a result of making the utensil 10 with a flexible material, these flat portions 19A may flex when the pockets are squeezed together, thereby further enhancing the gripping capability of the utensil.

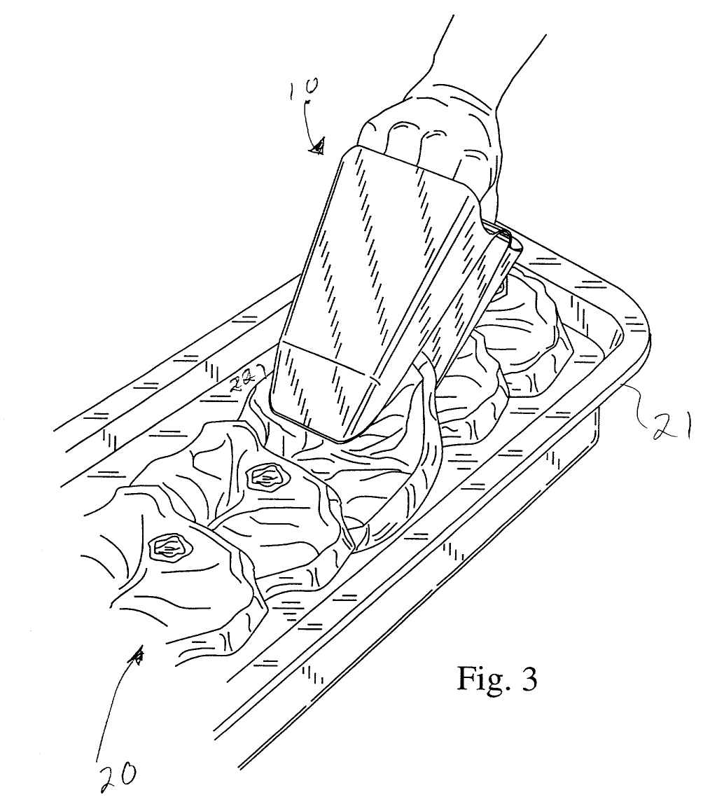

FIG. 3 illustrates the utensil 10 in use to handle meat 20 in a pan 21, for example, in a grocer's display case. The user simply inserts their hand into the pockets 11 and 12, and by the use of a squeezing pressure between the thumb and forefingers, is able to manipulate a piece 22 of the meat 20.

Accordingly, it has been shown that the utensil 10 enables a user to manipulate sensitive material without bringing bare hands into contact with the material. Moreover, the utensil 10 is easy to put on or take off, unlike a pair of Latex gloves. The utensil 10 is easy to slip on and off either a right hand or a left hand which allows for quick changing and multi-tasking by the user. This increases productivity because multiple utensils 10 can be used and put on and taken off easily for each task in the preparation and serving of food.

The utensil 10 may be color coded in order to reduce the chances of cross contamination with other food products. For example, one possible color scheme is to color one group of utensils 10 yellow for use in handling poultry, to color another group green for fruits or vegetables, to color still another group red for raw meat, to color yet another group brown for cooked meat, another group white for dairy and pastry, to color another group blue for fish, and so forth. Other colors may be used for other uses. In a micro-electronic assembly line, different colors could be used in a similar manner for identifying tasks to be performed or material to be handled.

The utensil 10 is of simple, low-cost construction, and could thus be a disposable tool. On the other hand, when constructed of durable plastic, it may become a permanent tool. The simplicity of construction also makes the utensil 10 very easy to clean. However, for handling ultra-sensitive material, such as where cross contamination of food may occur, disposable plastic covers may be used with the utensil 10. Such a disposable plastic cover could be treated with an anti-microbial agent in order to resist the growth of bacteria. Moreover, the utensil 10 itself may be constructed of a plastic containing an anti-microbial agent for resisting the growth of bacteria and thereby reducing the possibility of cross contamination.

While an illustrative and presently preferred embodiment of the invention has been described in detail herein, it is to be understood that the inventive concepts may be otherwise variously embodied and employed and that the appended claims are intended to be construed to include such variations except insofar as limited by the prior art.

What is claimed is:

1. A utensil for handling sensitive material comprising:
a) a pair of opposing pockets for receiving a user's hand, wherein each of the pockets has an open end and a closed end; and,
b) a hinge joining together the pair of pockets at the open ends thereof, wherein the hinge biases the pockets in a closed position such that the pair of pockets are separable by the user for gripping the sensitive material at the closed ends of the pockets.

2. The utensil as in claim 1 wherein the pair of pockets and hinge are made from a flexible plastic material having memory.

3. The utensil as in claim 1 wherein the pair of pockets and hinge are made from a flexible metal material having memory.

4. The utensil as in claim 1 wherein the pair of pockets and hinge are made from a flexible paper material having memory.

5. The utensil as in claim 1 made of a color indicating a task designation.

6. The utensil as in claim 1 wherein opposing surfaces of the pair of pockets are flat.

7. The utensil as in claim 6 wherein the opposing surfaces are bent inward near the closed ends of the pair of pockets, thereby forming pincers that enable the handling of small articles.

8. A utensil for handling sensitive material comprising:
a) a pair of pockets for receiving a user's hand and being formed of a flexible material, each of the pockets having an open end, a closed end and two sides, wherein one side is flat and arranged in opposition to the other side thereby forming opposing sides; and,
b) a hinge for joining together the pair of pockets at the opposing sides and at the open ends of the pockets, wherein the hinge biases the pockets in a closed position such that the pair of pockets are separable by the user for gripping the sensitive material with the opposing sides at the closed ends of the pockets.

9. The utensil as in claim 8 wherein the opposing sides are bent inward towards one another near the closed ends of the pair of pockets forming pincers that enable handling of small articles.

10. The utensil as in claim 8 wherein the pair of pockets and hinge are made from a flexible plastic material having memory.

11. The utensil as in claim 8 wherein the pair of pockets and hinge are made from a flexible metal material having memory.

12. The utensil as in claim 8 wherein the pair of pockets and hinge are made from a flexible paper material having memory.

13. The utensil as in claim 8 made of a color indicating a task designation.
